(19) United States
(12) Patent Application Publication
(10) Pub. No.: US 2025/0325418 A1
(43) Pub. Date: Oct. 23, 2025

(54) Gwen: Integrated Sanitary Napkin with Attached Wipe Package for Enhanced Hygiene

(71) Applicant: Felecia Williams, York, PA (US)

(72) Inventor: Felecia Williams, York, PA (US)

(21) Appl. No.: 18/638,751

(22) Filed: Apr. 18, 2024

(51) Int. Cl. A61F 13/551; A61F 13/472; A61F 13/51

(52) U.S. Cl. CPC A61F 13/55145; A61F 13/472; A61F 13/51; A61F 2013/51047; A61F 2013/5109

(57) ABSTRACT

The present invention discloses a novel sanitary napkin (pad) with an attached wipe package designed to improve feminine hygiene during menstruation. The integrated design allows females to conveniently clean themselves during pad changes, addressing a significant hygiene disconnect often experienced in such situations. The inventor seeks to claim the unique concept and implementation of this invention.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

Embodiments disclosed herein generally relate to menstrual pads or sanitary napkins. More particularly, the invention relates to an Integrated sanitary napkin with an attached wipe package for enhanced hygiene.

### Description of the Related Art

Sanitary pads, also known as menstrual pads or sanitary napkins, are absorbent products worn by individuals, primarily females, to manage menstrual flow. Historically, various materials such as cloth, wool, and grass have been used for menstrual hygiene purposes. However, the modern sanitary pad as we know it today emerged in the late 19th and early 20th centuries, with disposable pads becoming widely available in the mid-20th century. Since then, sanitary pads have undergone significant advancements in design, materials, and functionality to enhance comfort, absorbency, and convenience for users.

The global market for sanitary pads has experienced steady growth over the years, driven by factors such as increasing awareness of menstrual hygiene, growing female population, and rising disposable income levels. According to market research reports, the global sanitary pads market was valued at over $20 billion in 2020 and is projected to continue growing at a compound annual growth rate (CAGR) of around 6% from 2021 to 2026. Asia-Pacific region, particularly countries like India and China, constitutes a significant portion of the global sanitary pads market due to large population size and increasing adoption of menstrual hygiene products.

Innovations in sanitary pads are driven by various factors, including consumer preferences, technological advancements, regulatory requirements, and sustainability concerns. Key requirements for modern sanitary pads include high absorbency, comfort, breathability, leakage protection, odor control, and skin-friendliness. Additionally, there is a growing demand for eco-friendly and biodegradable options to address environmental concerns associated with disposable menstrual products. Innovations in materials, such as plant-based fibers and bioplastics, as well as advancements in manufacturing processes, are crucial to meeting these evolving requirements and improving the overall user experience.

The future of sanitary pads is expected to be characterized by continued innovation and diversification to cater to the diverse needs and preferences of consumers. Key trends shaping the future of the industry include:

Sustainability: Increased focus on eco-friendly and biodegradable materials to reduce environmental impact and meet consumer demand for sustainable products.

Technology Integration: Integration of technology, such as sensors and smart fabrics, to enhance functionality and provide real-time monitoring of menstrual health.

Customization: Customized products tailored to individual preferences and menstrual needs, including size, absorbency level, and comfort features.

Accessibility and Affordability: Efforts to improve access to menstrual hygiene products, particularly in low-income and underserved communities, through initiatives such as subsidized pricing and menstrual education programs.

Menstrual Health Awareness: Continued efforts to destigmatize menstruation and promote menstrual health education to empower individuals with knowledge and resources for managing their menstrual cycles effectively.

By looking at prior art multiple innovations have been seen in similar domain. For instance, a U.S. Pat. No. 10,123,539 B2, which describes an absorbent structure for sanitary napkins. This patent details a multi-layered design intended to enhance fluid management and user comfort by incorporating layers with different absorbency and flexibility properties. By optimizing the arrangement of materials, this invention aims to provide superior performance during menstruation.

Another notable patent, U.S. Pat. No. 9,732,280 B2, addresses the issue of odor control in sanitary napkins. This patent introduces a napkin design equipped with odor-absorbing materials and a breathable outer layer to mitigate unpleasant odors and enhance user comfort. By incorporating these features, the invention aims to improve the overall user experience and confidence during menstruation.

In the realm of sustainability, U.S. Pat. No. 3,165,482 B1 discloses a biodegradable sanitary pad designed to reduce environmental impact. This patent highlights the use of natural and biodegradable materials in the construction of the pad, offering an eco-friendly alternative to conventional disposable pads. With growing concerns about environmental sustainability, inventions like this play a crucial role in promoting more environmentally conscious menstrual hygiene practices.

Innovations in hygiene and cleanliness are also evident in patents such as CN Patent 103,784,255 B, which describes a self-cleaning sanitary napkin with antibacterial properties. This invention features a surface coating or treatment designed to inhibit bacterial growth and maintain hygiene during use. By incorporating antibacterial technology, this patent aims to enhance the overall cleanliness and hygiene of menstrual hygiene products.

Furthermore, U.S. Pat. No. 9,304,264 B2 introduces a reusable menstrual pad with adjustable absorbency levels. This patent outlines a pad design that includes removable inserts or layers, allowing users to customize the absorbency level based on their individual needs. This innovation offers a sustainable and cost-effective alternative to disposable pads while providing users with greater flexibility and control over their menstrual care routine.

The current innovation aims to provide advancement in a similar domain by providing a sanitary napkin (pad) featuring an attached wipe package integrated into its packaging. This innovative design enables females to access cleansing wipes directly from the pad packaging during menstrual changes, providing a convenient solution for maintaining hygiene and freshness.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

It is a general object of the present invention to provide a comprehensive solution for feminine hygiene by integrating a sanitary napkin (pad) with an attached wipe package, offering convenience and ease of use during menstrual periods.

It is also the objective of the invention to address the hygiene disconnect experienced by females during menstrual changes, ensuring that individuals have the option to clean themselves effectively with the attached wipes, enhancing overall cleanliness and comfort.

It is further the objective of the invention to provide an innovative concept and idea behind the integrated sanitary napkin with attached wipe package, recognizing the potential benefits and value it brings to menstrual hygiene practices.

It is also the objective of the invention to improve efficiency in menstrual care routines by securely attaching the wipes to the pad packaging, eliminating the need for separate packaging and accessories, and streamlining the process of accessing and using cleansing wipes.

It is another object to enhance the user experience and promote better hygiene practices by offering a convenient and hygienic solution for maintaining freshness and cleanliness during menstrual periods.

Other aspects, advantages and novel features of the present invention will become apparent from the detailed description of the invention when considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 shows the technical view of the assembly incorporating the embodiments of the invention.

FIG. 2 shows the product design of the assembly incorporating the embodiments of the invention.

FIG. 3 shows the pad with an attached wipe incorporating the embodiments of the invention.

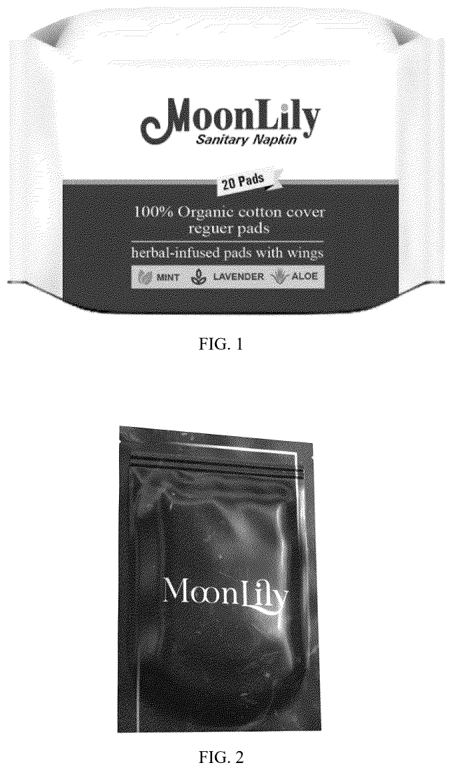

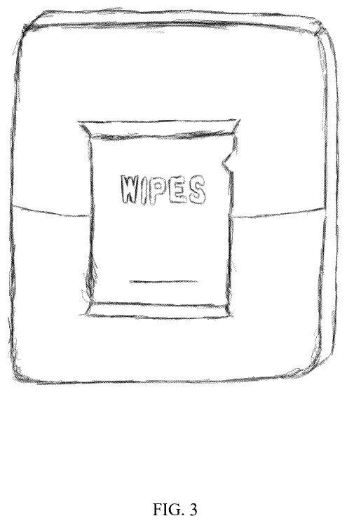

The apparatus and method components have been represented where appropriate by conventional symbols in the drawings, showing only those specific details

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present invention is directed to provide a sanitary napkin (pad) with an attached wipe package to address a prevalent hygiene gap faced by menstruating individuals

As per its preferred embodiments, the groundbreaking product presented is a transformative advancement in menstrual hygiene, combining a sanitary napkin (pad) with an attached wipe package to address a prevalent hygiene gap faced by menstruating individuals. Traditionally, during menstrual changes, users often encounter challenges in maintaining cleanliness and freshness. This innovative solution integrates cleansing wipes directly into the pad packaging, providing users with immediate access to a convenient hygiene option, thereby enhancing comfort and confidence during menstruation.

As per its preferred embodiments, the inventive product is to offer a holistic and efficient solution to the hygiene challenges associated with menstrual care. By seamlessly integrating the wipe package with the pad, this innovative product aims to streamline the menstrual hygiene routine, offering users a practical and user-friendly method for maintaining hygiene. By eliminating the need for separate products or accessories, the product optimizes the overall user experience and promotes better hygiene practices.

As per its preferred embodiments, the product provides a unique concept and design, recognizing its potential value in revolutionizing menstrual hygiene practices. By securing patent protection, the product aims to establish recognition and exclusivity for its groundbreaking contribution to feminine hygiene products. This commitment to innovation underscores a dedication to advancing the field of menstrual hygiene and addressing the specific needs of menstruating individuals.

As per its preferred embodiments the wipes are securely attached to the pad packaging, ensuring optimal efficiency and accessibility. This integrated design not only eliminates the risk of misplacement or loss but also enhances user confidence by providing a reliable and convenient hygiene solution during menstrual changes. Moreover, the attachment of wipes to the pad packaging promotes a sense of cleanliness and well-being throughout the menstrual cycle, enhancing overall comfort and hygiene.

As per its preferred embodiments the product represents a significant leap forward in menstrual hygiene products, offering a practical and innovative solution to a common hygiene concern. By addressing the hygiene disconnect experienced by menstruating individuals during pad changes, this product enhances comfort, confidence, and overall hygiene, ultimately improving the menstrual experience for users.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. A sanitary napkin with an absorbent structure comprising multiple layers of varying absorbency and flexibility properties.

2. The sanitary napkin of claim 1, wherein the absorbent structure includes a top layer designed for rapid fluid acquisition and distribution.

3. The sanitary napkin of claim 1, further comprising a core layer with enhanced absorbency to effectively capture and retain menstrual fluid.

4. The sanitary napkin of claim 1, further equipped with odor-absorbing materials integrated into the absorbent structure to mitigate unpleasant odors during use.

5. The sanitary napkin of claim 1, wherein the odor-absorbing materials are incorporated into one or more layers of the absorbent structure.

6. The sanitary napkin of claim 1, further developed from biodegradable sanitary pad comprising natural and biodegradable materials to reduce environmental impact and promote sustainability.

7. The sanitary napkin of claim 1, wherein the materials used in the construction of the pad are sourced from renewable resources.

8. The sanitary napkin of claim 1, wherein the napkin with antibacterial properties, comprising a surface coating or treatment designed to inhibit bacterial growth and maintain hygiene.

9. The sanitary napkin of claim 1, wherein the antibacterial surface coating is applied to one or more layers of the napkin.

10. The sanitary napkin of claim 1, wherein the reusable menstrual pad with adjustable absorbency levels, featuring removable inserts or layers to customize absorbency based on individual preferences.

11. The sanitary napkin of claim 1, wherein the removable inserts or layers are made from absorbent materials such as cotton or bamboo.

12. The sanitary napkin of claim 1, wherein the sanitary napkin comprises a breathable outer layer to enhance comfort and airflow during use.

13. The sanitary napkin of claim 1, wherein the breathable outer layer is made from a moisture-wicking material to keep the skin dry and comfortable.

14. The sanitary napkin of claim 1, wherein comprises of a combination of natural and synthetic fibers to optimize absorbency and fluid management.

15. The sanitary napkin of claim 1. wherein the natural fibers are derived from sources such as cotton, bamboo. or wood pulp. and the synthetic fibers are selected for their absorbency and durability properties.
